(19) United States
(12) Patent Application Publication
(10) Pub. No.: US 2022/0265153 A1
(43) Pub. Date: Aug. 25, 2022

(54) BIOLOGICAL INFORMATION GATHERING SYSTEM AND WEARABLE DEVICE

(71) Applicant: connectFree Corporation, Kyoto-shi, Kyoto (JP)

(72) Inventor: Kristopher Andrew TATE, Kyoto-shi, Kyoto (JP)

(73) Assignee: connectFree Corporation, Kyoto-shi, Kyoto (JP)

(21) Appl. No.: 17/631,211

(22) PCT Filed: Jul. 28, 2020
(86) PCT No.: PCT/JP2020/028857

§ 371 Date: Jan. 28, 2022

(30) Foreign Application Priority Data
Jul. 30, 2019 (JP) 2019-139861

(51) Int. Cl. A61B 5/0245; A61B 5/00; G16H 10/60

(52) U.S. Cl. CPC A61B 5/0245; A61B 5/6829; A61B 5/6887; G16H 10/60

(57) ABSTRACT

A biological information collection system is for collecting biological information of a user. The biological information collection system includes: a sensing device that is electrically connected to a member with which the user comes into contact and senses biological information of the user. A wearable device is placed in contact with a part of the user and stores the biological information sensed by the sensing device. A data server collects the biological information stored in the wearable device.

### TECHNICAL FIELD

The present disclosure relates to a biological information collection system and a wearable device.

### BACKGROUND ART

With the development of medical care, the average life expectancy is increasing year by year. In such a longevity society, it is preferable to detect some kind of defect and take preventive measures before getting sick. To deal with such a problem, there is a demand for a mechanism capable of easily measuring the health condition of each person.

For example, JP 2019-97757 A (Patent Document 1) discloses a biological information measuring device that can easily measure fluctuations in the blood pressure of a subject based on an image signal obtained by imaging a predetermined part of the subject.

### CITATION LIST

### Patent Document

Patent Document 1: JP 2019-97757 A

### SUMMARY OF THE INVENTION

### Problem to be Solved by the Invention

According to the biological information measuring device disclosed in Patent Document 1 described above, fluctuations in the blood pressure of a subject can be measured in a non-contact manner as long as imaging is possible. However, it is difficult to realize an environment in which imaging is always possible in daily life.

It is an object of the present disclosure to provide a solution that enables continuous collection of biological information in daily life.

### Means for Solving Problem

According to an aspect of the present disclosure, a biological information collection system for collecting biological information of a user is provided. The biological information collection system includes: a sensing device that is electrically connected to a member with which the user comes into contact and senses biological information of the user; a wearable device that is placed in contact with a part of the user and stores the biological information sensed by the sensing device; and a data server that collects the biological information stored in the wearable device.

The wearable device may acquire biological information from the sensing device by using a part of the user's body as a conductor.

The sensing device may be placed in public transportation.

The wearable device may be mounted as a part of shoes.

The data server may provide a report showing an amount of exercise or health condition of each user based on the collected biological information.

According to another aspect of the present disclosure, a wearable device worn by a user and used for collecting biological information of the user is provided. The wearable device includes a conductive member placed so as to be in contact with a part of the user and a controller electrically connected to the conductive member. The controller includes a memory for storing the biological information of the user and a communication unit for transmitting the biological information stored in the memory to an outside. The wearable device is mounted as a part of shoes.

### Effect of the Invention

According to the present disclosure, it is possible to provide a solution that enables continuous collection of biological information in daily life.

### BRIEF DESCRIPTION OF DRAWINGS

FIG. 1 is a schematic diagram showing an example of collecting and utilizing biological information in a biological information collection system according to the present embodiment;

FIG. 2 is a schematic diagram showing an example of the cross-sectional structure of a smart shoe used in the biological information collection system according to the present embodiment;

FIG. 3 is a schematic diagram showing a hardware configuration example of the smart shoe used in the biological information collection system according to the present embodiment;

FIG. 4 is a schematic diagram showing a configuration example of a data server configuring the biological information collection system according to the present embodiment;

FIG. 5 is a diagram for explaining a configuration example for collecting user's biological information while traveling by public transportation in the biological information collection system according to the present embodiment;

FIG. 6 is a diagram showing a sequence between smart shoes and a sensing device for collecting the biological information of the user shown in FIG. 5;

FIG. 7 is a diagram showing a sequence related to biological information transmission of the smart shoes in the biological information collection system according to the present embodiment;

FIG. 8 is a diagram for explaining an example of a service of health management and cooperation with a medical institution provided by the biological information collection system according to the present embodiment;

FIG. 9 is a diagram showing an example of a life report provided in the service shown in FIG. 8;

FIG. 10 is a diagram showing another example of the life report provided in the service shown in FIG. 8;

FIG. 11 is a diagram showing an example of a health management report provided in the service shown in FIG. 8;

FIG. 12 is a diagram for explaining an example of a service for cooperation with an insurance company provided by the biological information collection system according to the present embodiment; and

FIG. 13 is a diagram for explaining an example of additional functions of the smart shoes used in the biological information collection system according to the present embodiment.

### MODE(S) FOR CARRYING OUT THE INVENTION

An embodiment according to the present disclosure will be described in detail with reference to the diagrams. In addition, the same or corresponding portions in the diagrams are denoted by the same reference numerals, and the description thereof will not be repeated.

<A. Biological information collection system 1>

First, a biological information collection system 1 according to the present embodiment will be described. The biological information collection system 1 collects the biological information of a user. More specifically, the biological information collection system 1 collects biological information emitted from the user and provides various services based on the collected biological information.

In this specification, “biological information” is a term including various kinds of information generated by the activity of the user. More specifically, “biological information” can include information regarding the activity of the user (for example, the number of steps, walking distance, time traveled, time at rest, and calories burned) as well as various kinds of physiological and anatomical information generated by the user (living body) (for example, blood pressure, pulse rate, heart rate, and respiratory rate).

In the biological information collection system 1, biological information is collected through a wearable device worn by the user. In the present embodiment, as an example of the wearable device, shoes having necessary functions (hereinafter, also referred to as “smart shoes”) are mentioned. That is, a smart shoe in which a wearable device is mounted as a part of the shoe will be described as an example. In addition, the “wearable device” according to the present invention is not limited to shoes, but includes any device that the user can wear.

In addition, in the biological information collection system 1, a form is naturally assumed in which the wearable device is combined with another device instead of collecting biological information only by the wearable device.

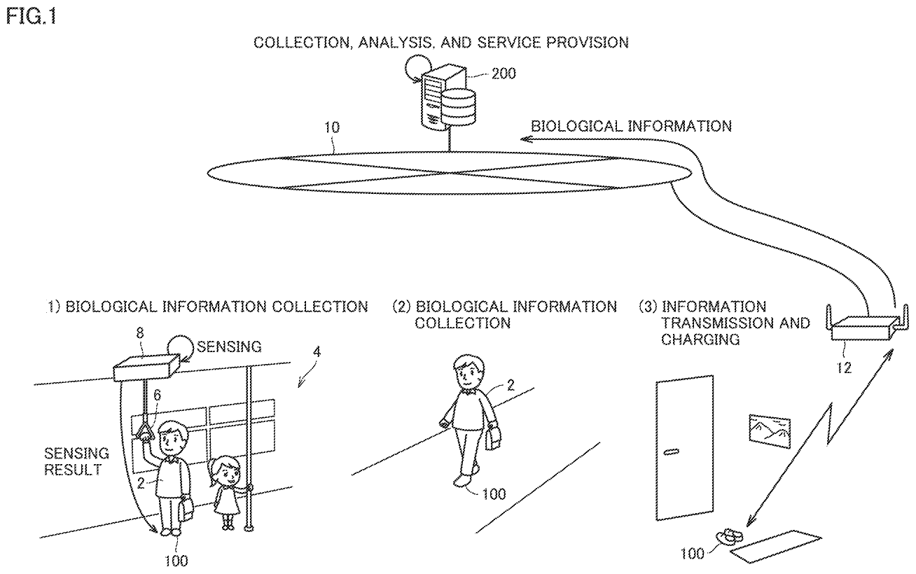

FIG. 1 is a schematic diagram showing an example of collecting and utilizing biological information in the biological information collection system 1 according to the present embodiment. Referring to FIG. 1, the biological information collection system 1 includes a data server 200 connected to a network 10, such as the Internet. The data server 200 collects biological information regarding a user 2, analyzes the collected biological information, and provides various services based on the analysis result (collection, analysis, and service provision).

Smart shoes 100 worn by the user 2 are used to collect biological information from the user 2. Typically, the smart shoes 100 have a function of directly or indirectly collecting biological information and a function of transmitting the collected biological information.

In the biological information collection system 1, a device for sensing the biological information of the user 2 may be prepared in a facility or device that the user 2 uses on a daily basis. For example, in the vehicle interior 4 of public transportation, such as a bus or a train, a sensing device 8 for sensing the biological information of the user 2 through a hanging strap 6 may be provided. For example, by placing a conductive material on the surface of the hanging strap 6, the sensing device 8 can sense the pulse rate, the heart rate, and the like of the user 2 ((1) biological information collection). Thus, the sensing device 8 is electrically connected to a member (for example, the hanging strap 6), with which the user 2 comes into contact, to sense the biological information of the user 2.

The biological information (sensing result) sensed by the sensing device 8 may be transmitted to the smart shoes 100 worn by the user 2. Alternatively, the sensing device 8 may acquire identification information from the smart shoes 100 worn by the user 2 and transmit the sensing result and the identification information to the data server 200.

In the biological information collection system 1, the smart shoes 100, which are a wearable device, can also sense the biological information of the user 2. For example, the smart shoes 100 can sense the number of steps, walking distance, time traveled, time at rest, and calories burned of the user 2. The result obtained by the sensing of the smart shoes 100 (sensing result) may be temporarily stored in the smart shoes 100, or may be transmitted from the smart shoes 100 to the data server 200 each time.

After the user 2 returns to his or her home and takes off the smart shoes 100, various kinds of information stored in the smart shoes 100 may be transmitted to the data server 200. At this time, the smart shoes 100 may be charged ((3) information transmission and charging). Typically, the smart shoes 100 transmit the stored biological information and the like to the data server 200 through an access point 12 connected to the network 10.

As described above, the smart shoes 100, which are an example of the wearable device, are placed in contact with a part of the user 2 (for example, the feet of the user 2) and stores the biological information sensed by the sensing device.

Details of various services provided by the data server 200 based on the biological information collected for each user will be described later.

<B. Configuration Example of Smart Shoes 100>

Next, a configuration example of the smart shoes 100 used in the biological information collection system 1 according to the present embodiment will be described. The smart shoes 100 are an example of a wearable device, and are worn by a user and used to collect biological information of the user.

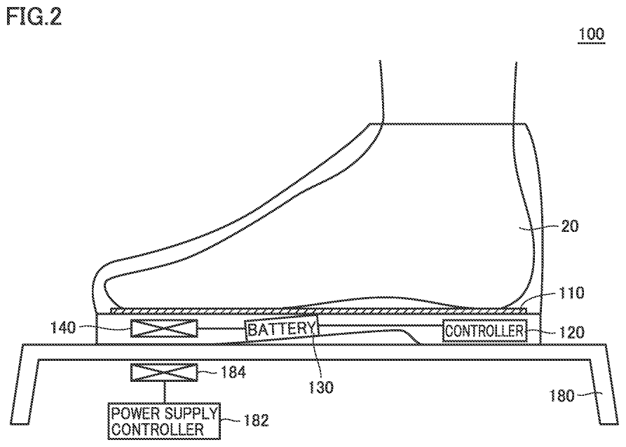

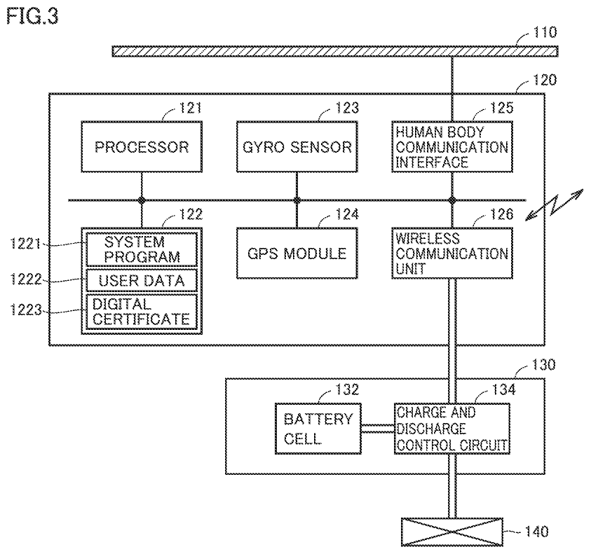

FIG. 2 is a schematic diagram showing an example of the cross-sectional structure of the smart shoe 100 used in the biological information collection system 1 according to the present embodiment. FIG. 3 is a schematic diagram showing a hardware configuration example of the smart shoe 100 used in the biological information collection system 1 according to the present embodiment.

Referring to FIG. 2, the smart shoe 100 includes a conductive member 110 placed at a position in contact with the foot 20 of the user 2. The conductive member 110 is placed so as to be in contact with a part of the user 2 and is electrically connected to a controller 120. The controller 120 makes electrical contact with the user 2 through the conductive member 110, and transmits and receives electrical signals by using a part of the body of the user 2 as a conductor (medium). For example, when the user 2 comes into contact with the hanging strap 6 (FIG. 1), the controller 120 can also communicate with the sensing device 8 (FIG. 1).

Electric power is supplied from a battery 130 to the controller 120. A power receiving coil 140 is electrically connected to the battery 130.

After the user 2 returns home and takes off the smart shoes 100, the smart shoes 100 are placed at a station 180 at home. The station 180 has a power supply coil 184 at a position that can face the power receiving coil 140. Electric power is supplied from a power supply controller 182 to the power supply coil 184.

Electric power is supplied from the power supply controller 182 to the battery 130 through electromagnetic coupling between the power supply coil 184 and the power receiving coil 140. By the electric power supplied in this manner, the battery 130 is charged.

Wireless communication may be performed by using the electromagnetic coupling between the power supply coil 184 and the power receiving coil 140.

Referring to FIG. 3, the controller 120 electrically connected to the conductive member 110 includes a processor 121, a memory 122, a gyro sensor 123, a GPS module 124, a human body communication interface 125, and a wireless communication unit 126.

The processor 121 may be, for example, a CPU (Central Processing Unit) or an MPU (Micro Processing Unit), and realizes various functions provided by the smart shoes 100 by reading and executing a program or the like stored in the memory 122.

The memory 122 has a non-volatile storage region and a volatile storage region. A system program 1221, user data 1222, a digital certificate 1223, and the like are stored in the non-volatile storage region. The system program 1221 includes computer-readable instructions for realizing the functions of the smart shoes 100. The user data 1222 includes biological information acquired by an arbitrary method. The digital certificate 1223 includes identification information for identifying the smart shoes 100. Thus, the memory 122 may store the biological information of the user 2.

The gyro sensor 123 senses the posture, moving speed, and the like of the smart shoes 100 based on the acceleration or the like generated in the smart shoes 100. The GPS module 124 acquires the position information of the smart shoes 100.

In addition, in FIG. 3, the gyro sensor 123 and the GPS module 124 are exemplified as sensing devices of the smart shoes 100, but the invention is not limited thereto, and any sensing device (for example, a piezoelectric sensor, an acceleration sensor, a temperature sensor, a humidity sensor, and an illuminance sensor) may be adopted.

The human body communication interface 125 is electrically connected to the conductive member 110, and transmits and receives electrical signals through the conductive member 110 by using a part of the body of the user 2 as a conductor (medium).

The wireless communication unit 126 transmits and receives a wireless signal to and from the access point 12

(FIG. 1) or the like. Typically, the wireless communication unit 126 transmits the biological information stored in the memory 122 to the outside.

The battery 130 includes a battery cell 132 and a charge and discharge control circuit 134. The battery cell 132 is a device for storing electric power, and it is preferable to use a flexible material. The charge and discharge control circuit 134 controls charging and discharging of the battery cell 132. Specifically, the charge and discharge control circuit 134 charges the battery cell 132 with a part or entirety of the electric power supplied from the power receiving coil 140, and supplies electric power discharged from the battery cell 132 to the controller 120.

<C. Configuration Example of Data Server 200>

Next, a configuration example of the data server 200 configuring the biological information collection system 1 according to the present embodiment will be described. Typically, the data server 200 is realized by using one or more general-purpose computers.

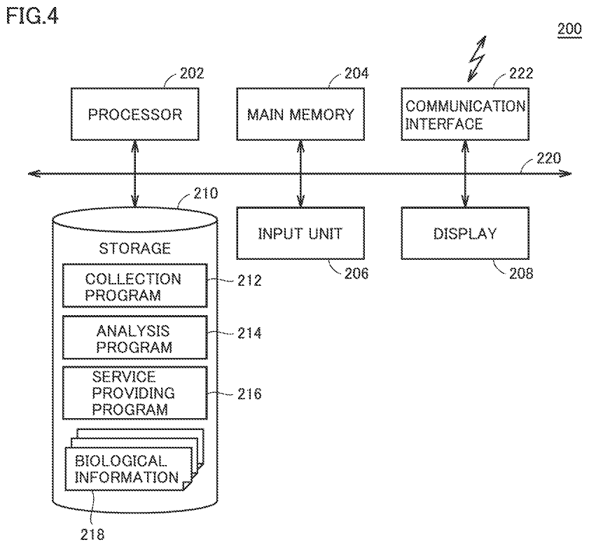

FIG. 4 is a schematic diagram showing a configuration example of the data server 200 configuring the biological information collection system 1 according to the present embodiment. Referring to FIG. 4, the data server 200 includes one or more processors 202, a main memory 204, an input unit 206, a display 208, a storage 210, and a communication interface 222 as main components. These components are connected to each other through an internal bus 220.

The processor 202 may be, for example, a CPU or a GPU (Graphics Processing Unit). A plurality of processors 202 may be arranged, or the processor 202 having a plurality of cores may be adopted.

The main memory 204 is a volatile storage device, such as a DRAM (Dynamic Random Access Memory) or an SRAM (Static Random Access Memory). The storage 210 is a non-volatile storage device, such as a hard disk or an SSD (Solid State Drive), and stores various programs or various kinds of data to be executed by the processor 202. Among the programs stored in the storage 210, a designated program code is loaded to the main memory 204, and the processor 202 sequentially executes computer-readable instructions included in the program code loaded to the main memory 204 to realize various functions described later.

Typically, a collection program 212 for collecting biological information of each user, an analysis program 214 for analyzing the collected biological information, a service providing program 216 for providing various services based on the analysis result, and collected biological information 218 are stored in the storage 210.

The input unit 206 receives an input operation of the user. The display 208 displays the processing result of the processor 202 and the like.

The communication interface 222 is in charge of data exchange with the smart shoes 100. The communication interface 222 may include, for example, an Ethernet (registered trademark) port for communication over the Internet.

The entirety or part of the data server 200 may be realized by using a hard-wired circuit such as an ASIC (Application Specific Integrated Circuit) in which a circuit corresponding to computer-readable instructions is provided. Alternatively, the entirety or part of the data server 200 may be realized by using a circuit corresponding to computer-readable instructions on an FPGA (field-programmable gate array). In addition, the entirety or part of the data server 200 may be realized by appropriately combining the processor 202, a main memory, an ASIC, an FPGA, and the like.

The data server 200 may further include a component for reading the stored program and the like from the non-transitory medium that stores the collection program 212, the analysis program 214, and the service providing program 216 that are computer-readable instructions. The medium may be, for example, an optical medium, such as a DVD (Digital Versatile Disc), or a semiconductor medium, such as a USB memory.

The collection program 212, the analysis program 214, and the service providing program 216 may not only be installed on the data server 200 through the medium, but also be provided from a distribution server on the network.

<D. Collection of Biological Information>

In the biological information collection system 1 according to the present embodiment, it is possible to collect biological information in daily life without making the user aware of it. Some of such biological information collection forms will be described.

(d1: Traveling by Public Transportation)

A configuration example in which the biological information of the user 2 is collected while traveling by public transportation, such as a bus or a train, will be described.

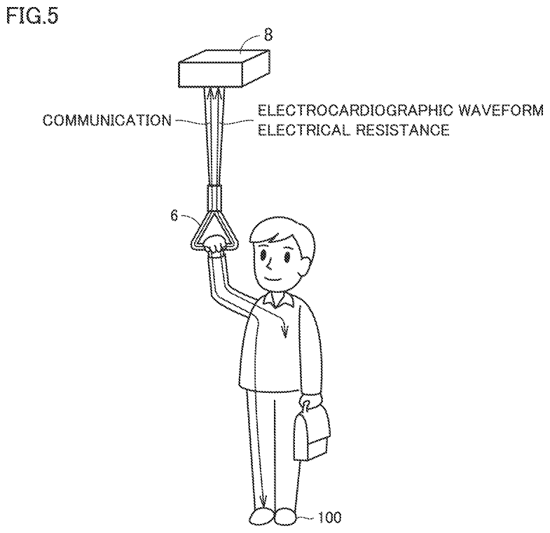

FIG. 5 is a diagram for explaining a configuration example for collecting user's biological information while traveling by public transportation in the biological information collection system 1 according to the present embodiment. Referring to FIG. 5, the sensing device 8 is placed in public transportation, such as a bus or a train. A conductive material is placed on the surface of the hanging strap 6, so that a conduction path is formed between the sensing device 8 and the user 2. In addition, a conduction path is formed between the smart shoes 100 and the user 2. Therefore, a conduction path can also be formed between the sensing device 8 and the smart shoes 100 worn by the user 2.

The sensing device 8 transmits and receives necessary information to and from the smart shoes 100 and senses the biological information of the user 2. For example, the pulse rate, the heart rate, and the like of the user 2 are sensed based on the electrocardiographic waveform of the user 2. In addition, the sensing device 8 may sense the body composition ratio of the user 2 as biological information based on the electrical resistance generated by the user 2.

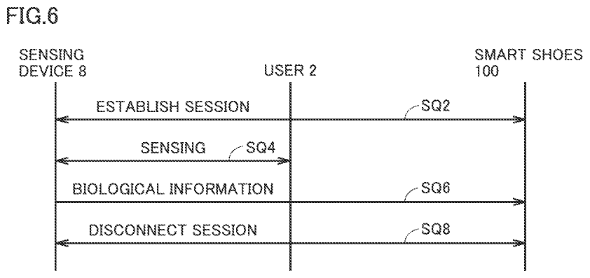

FIG. 6 is a diagram showing a sequence between the smart shoes 100 and the sensing device 8 for collecting the biological information of the user shown in FIG. 5. Referring to FIG. 6, when the user 2 touches the hanging strap 6, a conduction path is formed between the smart shoes 100 and the sensing device 8 through the user 2. Then, a session is established between the smart shoes 100 and the sensing device 8 according to a predetermined procedure (sequence SQ2).

Then, the sensing device 8 senses the biological information of the user 2 based on the electrical signal or the like acquired from the user 2 (sequence SQ4). The sensing device 8 transmits the sensed biological information (sensing result) to the smart shoes 100 (sequence SQ6). The smart shoes 100 store the transmitted biological information of the user 2.

Finally, the session between the smart shoes 100 and the sensing device 8 is disconnected (sequence SQ8).

The biological information sensed from the user 2 by the above sequence is stored in the smart shoes 100 worn by the user 2. As will be described later, the smart shoes 100 transmit the stored biological information to the data server 200 by using an arbitrary method.

In addition, although the configuration example of transmitting the biological information (sensing result) sensed by the sensing device 8 to the smart shoes 100 has been described in FIGS. 5 and 6, the sensing device 8 may directly transmit the sensing result to the data server 200. In this case, the data server 200 can associate the received sensing result with the user 2 by transmitting the identification information of the smart shoes 100, which is acquired when establishing a session between the sensing device 8 and the smart shoes 100, to the data server 200 together with the sensing result.

As described above, the smart shoes 100, which are an example of the wearable device, acquires biological information from the sensing device 8 by using a part of the body of the user 2 as a conductor.

The biological information of the user 2 can be collected while traveling by public transportation such as a bus or a train as described above. Therefore, it is possible to efficiently collect biological information without making the user 2 aware of it and without taking time for sensing.

(d2: Sensing of Biological Information Through an Object that the User 2 Touches on a Daily Basis)

In the configuration example shown in FIGS. 5 and 6 described above, the collection of biological information in public transportation such as a bus or a train has been exemplified. However, the invention is not limited thereto, and the biological information of the user 2 may be collected through an object that the user 2 touches in daily life.

For example, a conductive material may be placed on the entirety or part of a steering wheel of a vehicle so that the biological information is collected while the user 2 is driving the vehicle. Alternatively, the biological information of the user 2 may be collected through a doorknob.

In this manner, by making it possible to collect biological information through an object that the user 2 touches in daily life, it is possible to efficiently collect the biological information without making the user 2 aware of it and without taking time for sensing.

(d3: Smart Shoes 100)

In the configuration example shown in FIGS. 5 and 6 described above, a configuration example in which the sensing device 8 placed on the object touched by the user 2 in daily life collects biological information has been described. However, the smart shoes 100 itself may collect biological information.

Any sensing device can be placed on the smart shoes 100. Since the smart shoes 100 are worn by the user 2, it is possible to collect arbitrary information regarding the user 2 according to the placed sensing device.

FIG. 3 shows the smart shoes 100 including the gyro sensor 123. By using the gyro sensor 123, it is possible to collect the number of steps, walking posture, time traveled, time at rest, calories burned, and the like. In addition, by using the information from the GPS module 124, it is possible to collect information such as a walking distance or a walking speed.

In addition, for example, by placing a piezoelectric sensor or the like, it is possible to collect the force generated when the user 2 walks.

As described above, since the smart shoes 100 are in contact with the user 2, various kinds of biological information can be collected from the user 2. By adopting such a configuration, it is possible to efficiently collect biological information without making the user 2 aware of it and without taking time for sensing.

<E. Transmission of Biological Information>

Next, a configuration example for transmitting biological information stored in the smart shoes 100 to the data server 200 will be described.

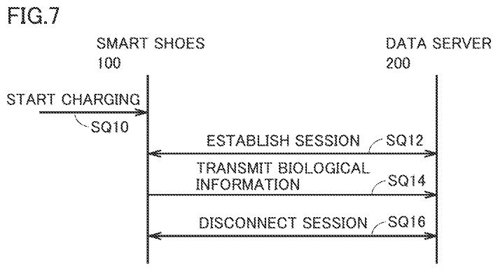

FIG. 7 is a diagram showing a sequence related to biological information transmission of the smart shoes 100 in the biological information collection system 1 according to the present embodiment. Referring to FIG. 7, the smart shoes 100 are placed at the station 180 at home, and when charging of the smart shoes 100 is started (sequence SQ10), a session is established between the smart shoes 100 and the data server 200 according to a predetermined procedure (sequence SQ12). Then, the smart shoes 100 transmit the stored biological information of the user 2 to the data server 200 (sequence SQ14). The smart shoes 100 transmit identification information indicating the smart shoes 100 or the user 2 wearing the smart shoes 100 together with the biological information. The data server 200 stores the transmitted biological information of the user 2. At this time, the data server 200 stores the biological information in association with the identification information from the smart shoes 100.

Finally, the session between the smart shoes 100 and the data server 200 is disconnected (sequence SQ16).

By the above sequences, the biological information sensed from the user 2 stored in the smart shoes 100 is transmitted to the data server 200.

<F. Examples of Utilization of Biological Information>

Next, an example of utilizing the biological information of the user 2 collected by the data server 200 will be described.

(f1: Health Management/Medical Institution)

As one of utilization examples, a service of health management for each user and cooperation with a medical institution based on biological information of each user collected in the data server 200 will be described.

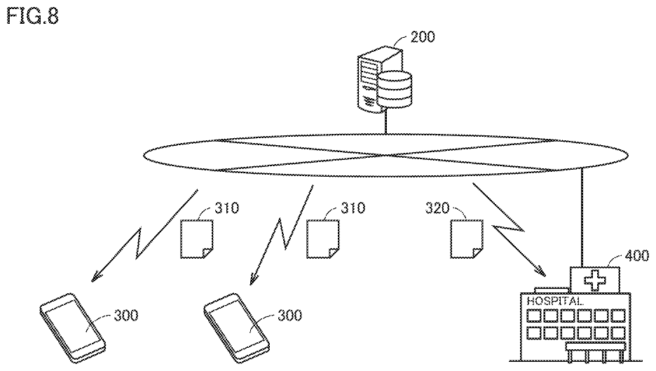

The data server 200 may provide a report showing the amount of exercise or health condition of each user based on the collected biological information. FIG. 8 is a diagram for explaining an example of a service of health management and cooperation with a medical institution provided by the biological information collection system 1 according to the present embodiment. Referring to FIG. 8, the data server 200 may transmit a life report 310 to a mobile terminal 300 or the like owned by the user 2 on a regular basis or at each event.

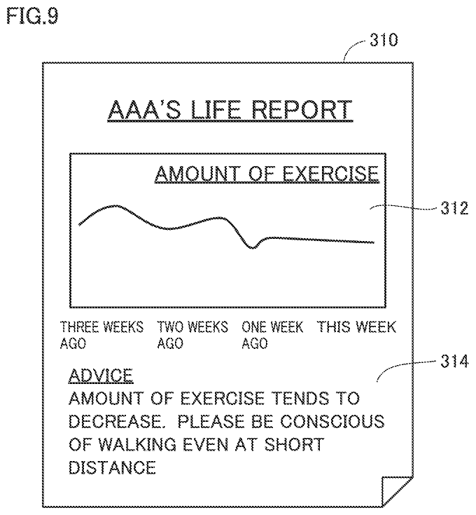

FIG. 9 is a diagram showing an example of the life report 310 provided in the service shown in FIG. 8. Referring to FIG. 9, the life report 310 includes, for example, a graph 312 showing the amount of exercise for a predetermined period and an advice 314 based on a change in the amount of exercise shown in the graph 312 or the like. The amount of exercise shown in the graph 312 may be, for example, a score calculated based on information regarding the activity of the user 2 (for example, the number of steps, walking distance, time traveled, time at rest, and calories burned). In addition, the amount of exercise may be calculated by any method using arbitrary biological information.

In addition, the life report 310 can include arbitrary information generated based on the biological information of the user 2 collected by the data server 200.

In addition, the destination to which the data server 200 provides the life report 310 can be arbitrarily set by the user 2. As a method of providing the life report 310, e-mail may be used or a Web-based method may be used. In addition, the life report 310 of the user 2 may be provided to other users (for example, when the user 2 is elderly, the child of the user 2) related to the user 2. By providing the life report 310 to other users, the health condition of the user 2 can be checked remotely.

In addition, the life report 310 including a game-like element may be provided.

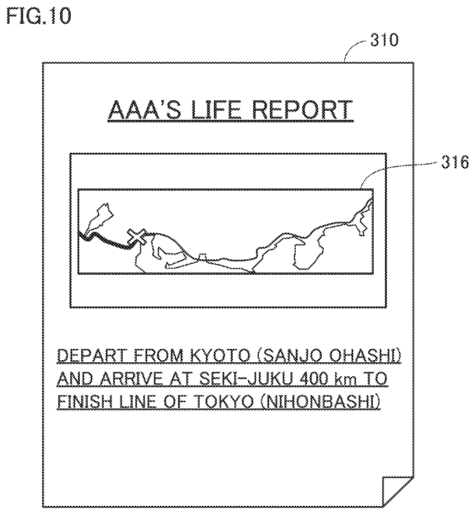

FIG. 10 is a diagram showing another example of the life report 310 provided in the service shown in FIG. 8. The life report 310 shown in FIG. 10 includes a map graphic 316 in which the walking distance of the user 2 is applied to the 53 Stations of the Tokaido.

By providing the life report 310 including such a game-like element, it is possible to motivate the user to walk more.

Referring to FIG. 8 again, the data server 200 may transmit a health management report 320 to a medical institution 400 registered in advance by the user 2 on a regular basis or at each event.

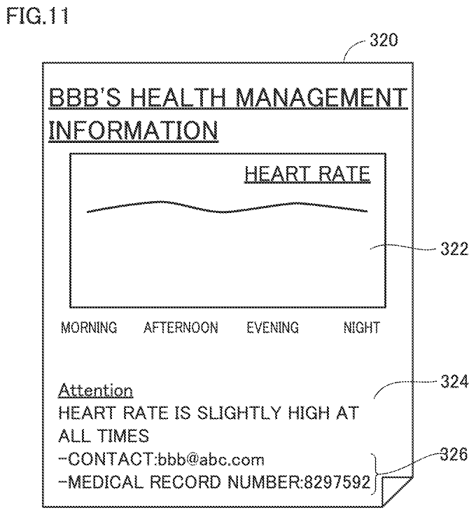

FIG. 11 is a diagram showing an example of the health management report 320 provided in the service shown in FIG. 8. Referring to FIG. 11, the health management report 320 includes, for example, a graph 322 showing the heart rate for a predetermined period and an attention message 324 based on the heart rate state shown in the graph 322 and the like. In addition, the health management report 320 may include contact information 326 of the user 2.

The attention message 324 is output by applying arbitrary criteria to the biological information collected in the data server 200. For example, for the attention message 324 for the heart rate shown in FIG. 11, predetermined statistical criteria may be applied, or criteria determined based on the history of the past heart rate of the user 2 may be applied.

In addition, the information included in the health management report 320 may be any information as long as the information is obtained from the biological information of the user 2 collected by the data server 200. In addition, the health management report 320 including a plurality of types of biological information may be adopted.

By providing such a health management report 320 to the medical institution 400 (or the attending physician in the medical institution 400) registered in advance by the user 2, it is possible to manage and monitor the health condition of the user 2 from the expert's point of view.

(f2: Insurance Company)

As another example of utilization, a service for providing an insurance company with the health condition of each user based on biological information of each user collected in the data server 200 will be described.

For example, when the user 2 applies for some kind of insurance, from the perspective of the insurance company to which the insurance is undertaken, it is rational to determine whether or not to accept the insurance application, an insurance premium, and the like based on the health condition of the user 2 who has applied for the insurance.

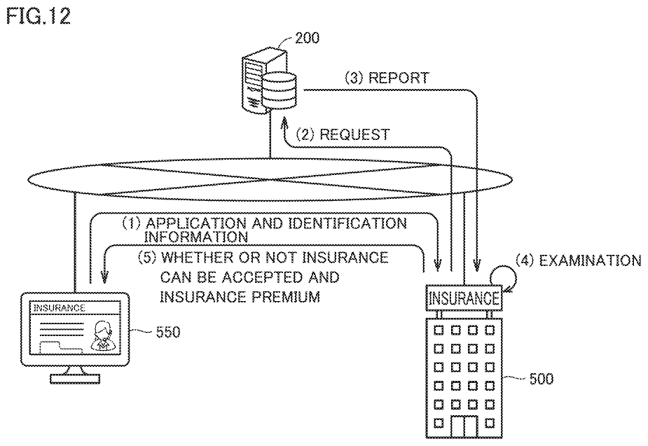

FIG. 12 is a diagram for explaining an example of a service for cooperation with an insurance company provided by the biological information collection system 1 according to the present embodiment. Referring to FIG. 12, an insurance company 500 (a server computer or a client computer) can transmit and receive data to and from the data server 200.

As a typical procedure, when the user 2 applies for insurance using a terminal device 550, the identification information of the user 2 is also input ((1) application and identification information). The identification information is information for identifying the biological information of the user 2 collected in the data server 200.

When the insurance company 500 receives the insurance application and the identification information from the user 2, the insurance company 500 transmits the received identification information to the data server 200 and makes a request for the biological information of the user 2 ((2) request). In response to the request from the insurance company 500, the data server 200 provides the biological information corresponding to the designated identification information to the insurance company 500 as a report ((3) report).

The insurance company 500 determines whether or not to accept the insurance application from the user 2 and the insurance premium when accepting the insurance application based on the report provided by the data server 200 ((4) examination).

Then, the insurance company 500 notifies the user 2 of whether or not the applied insurance can be accepted and the insurance premium (when the applied insurance can be accepted) ((5) whether or not the insurance can be accepted and insurance premium).

In this manner, by using the biological information of the user 2 in response to the insurance application from the user 2, it is possible to reasonably determine whether or not to accept the applied insurance and the insurance premium.

<G. Additional Functions>

Next, an example of the additional functions of the smart shoes 100 will be described. As described above, the smart shoes 100 transmit and receive electrical signals by using a part of the body of the user 2 wearing the smart shoes 100 as a conductor (medium). For example, an electronic key may be realized by using such transmission and reception of electrical signals (human body communication).

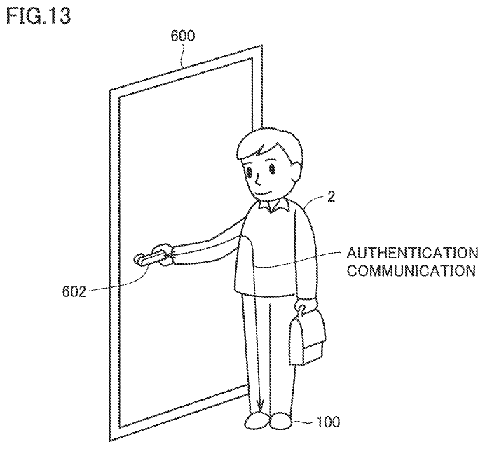

FIG. 13 is a diagram for explaining an example of additional functions of the smart shoes 100 used in the biological information collection system 1 according to the present embodiment. Referring to FIG. 13, when the user 2 wearing the smart shoes 100 touches a doorknob 602 of a door 600, a conduction path is formed between the smart shoes 100 and the doorknob 602 through the user 2.

The doorknob 602 has a built-in communication circuit for performing authentication communication with the smart shoes 100. When the authentication communication between the doorknob 602 and the smart shoes 100 is successful, the doorknob 602 is unlocked. In this manner, the smart shoes 100 can also be used as an electronic key.

In addition, when the smart shoes 100 are used as an electronic key, a certificate may be built in in advance to unlock the home key. For example, a certificate that allows you to enter a room reserved at the time of booking, such as a hotel, may be stored in the smart shoes 100.

In this manner, the smart shoes 100 can communicate with any object touched by the user 2 wearing the smart shoes 100, and it is possible to provide various functions and services by such communication.

<H. Other Forms>

In the above description, the smart shoes 100 having a configuration in which the conductive member 110, the controller 120, the battery 130, and the power receiving coil 140 are integrated with the shoes have been exemplified. However, the invention is not limited thereto, and the smart shoes 100 may be realized in any form. For example, an insole to be put into the shoe may be made to have a function and a configuration required for the smart shoes 100. By adopting such a mounting form as an insole, even when there are a plurality of shoes, the function of the biological information collection system according to the present embodiment can be realized by exchanging the insoles.

In addition, when the same user has a plurality of shoes, the identification information indicating the user may be held in common among the plurality of shoes. By sharing the identification information assigned to a specific user, the biological information of the user can be collected in the data server 200 regardless of which shoes the same user wears, so that a consistent service can be provided to the user.

<I. Advantages>

In the biological information collection system 1 according to the present embodiment, the biological information of the user is collected in daily life without the user being aware of it in particular. Various services can be provided based on the biological information collected in the data server.

In the biological information collection system 1 according to the present embodiment, since the biological information of the user is continuously collected in daily life, it is possible to provide various reports suitable for the health management of the user, and it can be easily realized to provide various reports to the medical institution where there is an attending physician or the like.

It should be considered that the embodiment disclosed is an example in all points and not restrictive. The scope of the invention is defined by the claims rather than the above description, and is intended to include all modifications within the scope and meaning equivalent to the claims.

### EXPLANATIONS OF LETTERS OR NUMERALS

1 BIOLOGICAL INFORMATION COLLECTION SYSTEM

2 USER

4 VEHICLE INTERIOR

6 HANGING STRAP

8 SENSING DEVICE

10 NETWORK

12 ACCESS POINT

20 FOOT

100 SMART SHOES

110 CONDUCTIVE MEMBER

120 CONTROLLER

121, 202 PROCESSOR

122 MEMORY

123 GYRO SENSOR

124 GPS MODULE

125 HUMAN BODY COMMUNICATION INTERFACE

126 WIRELESS COMMUNICATION UNIT

130 BATTERY

132 BATTERY CELL

134 CHARGE AND DISCHARGE CONTROL CIRCUIT

140 POWER RECEIVING COIL

180 STATION

182 POWER SUPPLY CONTROLLER

184 POWER SUPPLY COIL

200 DATA SERVER

204 MAIN MEMORY

206 INPUT UNIT

208 DISPLAY

210 STORAGE

212 COLLECTION PROGRAM

214 ANALYSIS PROGRAM

216 SERVICE PROVIDING PROGRAM

218 BIOLOGICAL INFORMATION

220 INTERNAL BUS

222 COMMUNICATION INTERFACE

300 MOBILE TERMINAL

310 LIFE REPORT

312, 322 GRAPH

314 ADVICE

316 MAP GRAPHICS

320 HEALTH MANAGEMENT REPORT

324 ATTENTION MESSAGE

326 CONTACT INFORMATION

400 MEDICAL INSTITUTION

500 INSURANCE COMPANY

550 TERMINAL DEVICE

600 DOOR

602 DOOR KNOB

1221 SYSTEM PROGRAM

1222 USER DATA

1223 DIGITAL CERTIFICATE

1-6. (canceled)

7. A system for collecting biological information of a user, comprising:
a sensing device configured to sense biological information of the user; and
a wearable device placed in contact with a part of the user and configured to store the biological information sensed by the sensing device;
wherein the sensing device is external to the wearable device and electrically connected to a member with which the user comes into contact.

8. The system according to claim 7, wherein
the wearable device acquires the biological information from the sensing device by using a part of the user's body as a conductor.

9. The system according to claim 7, wherein
the sensing device is arranged in public transportation.

10. The system according to claim 7, wherein
the wearable device is mounted as a part of shoes.

11. The system according to claim 7, further comprising:
a data server configured to collect the biological information stored in the wearable device.

12. The system according to claim 11, wherein
the data server provides a report showing an amount of exercise or health condition of each user based on the collected biological information.

13. The system according to claim 11, further comprising:
a computer arranged at an insurance company, wherein:
at the data server, the collected biological information is associated with identification information for identifying a user from whom the biological information is collected;
in response to an application for insurance from a user along with identification information of the user, the computer transmits the identification information to the data server and receives biological information corresponding to the identification information; and
the computer notifies the user of whether or not the applied insurance can be accepted.

14. The system according to claim 13, wherein
the computer further notifies the user of an insurance premium when the applied insurance can be accepted.

15. A method of collecting biological information of a user, comprising:
sensing, at a sensing device, biological information of the user; and
storing, at a wearable device placed in contact with a part of the user, the biological information sensed by the sensing device;
wherein the sensing device is external to the wearable device and electrically connected to a member with which the user comes into contact.

16. The method according to claim 15, further comprising:
acquiring, at the wearable device, the biological information from the sensing device by using a part of the user's body as a conductor.

17. The method according to claim 15, wherein
the sensing device is arranged in public transportation.

18. The method according to claim 15, wherein
the wearable device is mounted as a part of shoes.

19. The method according to claim 15, further comprising:
collecting, at a data server, the biological information stored in the wearable device.

20. The method according to claim 19, further comprising:
providing, at the data server, a report showing an amount of exercise or health condition of each user based on the collected biological information.

21. The method according to claim 19, further comprising:
providing a computer arranged at an insurance company;
associating, at the data server, the collected biological information with identification information for identifying a user from whom the biological information is collected;
transmitting, at the computer, the identification information to the data server in response to an application for insurance from a user along with identification information of the user;
receiving, at the computer, biological information corresponding to the identification information; and
notifying, at the computer, the user of whether or not the applied insurance can be accepted.

22. The method according to claim 13, further comprising:
notifying, at the computer, the user of insurance premium when the applied insurance can be accepted.
